(19) United States
(12) Patent Application Publication
(10) Pub. No.: US 2024/0416042 A1
(43) Pub. Date: Dec. 19, 2024

(54) A medicament container carrier of a medicament delivery device and a method of assembling a medicament delivery device medicament container assembly

(71) Applicant: SHL Medical AG, Zug (CH)

(72) Inventor: Ming-Ting Yin, Taoyuan City (TW)

(21) Appl. No.: 18/700,835

(22) PCT Filed: Oct. 6, 2022
(86) PCT No.: PCT/EP2022/077872

§ 371 Date: Apr. 12, 2024

(30) Foreign Application Priority Data
Oct. 19, 2019 (EP) 21203427.6

(51) Int. Cl. A61M 5/24

(52) U.S. Cl. CPC A61M 5/24; A61M 2005/2433; A61M 2207/00

(57) ABSTRACT

The present disclosure provides a medicament container carrier of a medicament delivery device, the medicament container carrier including a tubular body for receiving a medicament container, the tubular body extending along a longitudinal axis between a proximal open end and a distal open end, the tubular body including a fastener. The medicament container further includes a proximal cap with a tubular cap body. The tubular cap body extends along the longitudinal axis between a proximal end and a distal end. A slot extends on the tubular cap body from the proximal end to the distal end. The tubular cap body includes a ledge extending from an inner surface of the tubular cap body. The ledge is radial relative to the longitudinal axis, and the ledge is configured to support a shoulder part of the medicament container of the medicament delivery device. The proximal cap includes a counter fastener offset relative to the ledge in the direction of the longitudinal axis. The proximal cap is attached to the proximal end of the tubular body through an engagement between the fastener and the counter fastener.

### CROSS-REFERENCE TO RELATED APPLICATIONS

The present application is a U.S. National Phase Application pursuant to 35 U.S.C. § 371 of International Application No. PCT/EP2022/077872 filed Oct. 6, 2022, which claims priority to European Patent Application No. 21203427.6 filed Oct. 19, 2021. The entire disclosure contents of these applications are herewith incorporated by reference into the present application.

### TECHNICAL FIELD

The present disclosure generally relates to a medicament container carrier of a medicament delivery device and a method of assembling a medicament delivery device medicament container assembly, and particularly to a medicament container carrier comprising a proximal cap.

### BACKGROUND

Medicament delivery devices are often used for self-administration of a drug by puncturing of a patient's skin and the delivery of the drug via a needle. This process is preferably performed rapidly which requires that a significant force is applied to the medicament container by a drive mechanism. However, medicament containers can be fragile and when the force of the drive mechanism is applied, there is a significant risk that the fragile medicament container breaks.

For this reason, solutions for supporting the medicament container with a medicament container carrier that prevents the medicament container from damage when it is subject to the force from the drive mechanism is highly valued. Thus, a robust medicament container assembly that includes a medicament container carrier would be beneficial.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided with a medicament container carrier of a medicament delivery device, the medicament container carrier comprising: a tubular body for receiving a medicament container, the tubular body extending along a longitudinal axis between a proximal open end and a distal open end; the tubular body comprises a fastener; a proximal cap comprising a tubular cap body; the tubular cap body extends along the longitudinal axis between a proximal end and a distal end; a slot extends on the tubular cap body from the proximal end to the distal end; the tubular cap body comprises a ledge extending from an inner surface of the tubular cap body; the ledge is radial relative to the longitudinal axis; the ledge is configured to support a shoulder part of the medicament container of the medicament delivery device; the proximal cap comprises a counter fastener offset relative to the ledge in the direction of the longitudinal axis; and the proximal cap is attached to the proximal end of the tubular body through an engagement between the fastener and the counter fastener.

Preferably, according to another embodiment, the fastener is either a cut-out/recess or a hook; and the counter fastener is a hook if the fastener is a cut-out/recess, or the counter fastener is a cut-out/recess if the fastener is a hook.

Preferably, according to another embodiment, the fastener is the cut-out/recess; the cut-out/recess is arranged in a wall of the tubular body; the cut-out/recess is open in a direction transverse to the longitudinal axis; the counter fastener is the hook extending from the tubular cap body in the distal direction relative to the tubular cap body; and the proximal cap is attached to the proximal end of the tubular body through a fastener between the hook and the cut-out/recess.

Preferably, according to another embodiment, the cut-out/recess extends in a circumferential direction relative to the longitudinal axis between a first end and a second end; a ramp surface arranged at the first end; and the ramp surface inclines from an inside portion of the cut-out/recess to an outer edge of the cut-out/recess.

Preferably, according to another embodiment, the hook comprises a hook arm and a hook tip; the hook arm extends the direction of the longitudinal axis; the hook tip extends from an end of the hook arm in the direction transverse to the longitudinal axis; and the hook tip is arranged within the cut-out/recess.

Preferably, according to another embodiment, the hook tip extends in a circumferential direction between a first end and a second end; and the hook tip comprises a ramp surface angled to the hook arm and arranged at the first end.

Preferably, according to another embodiment, the engagement between the fastener and the counter fastener is a snap-fit engagement.

Preferably, according to another embodiment, the tubular body comprises a distal ledge extending from an inner surface of the tubular body.

Preferably, according to another embodiment, the distal ledge is an annular ledge.

Preferably, according to another embodiment, the tubular cap body comprises multiple ledges extending from the inner surface of the tubular cap body; and the multiple ledges are offset relative to one another in the direction of the longitudinal axis, so that the shoulders of medicament containers of the medicament delivery device with different lengths can be supported by one of the ledges.

Preferably, according to another embodiment, the tubular cap body has varied inner diameters, so that the shoulders of medicament containers of the medicament delivery device with different lengths and/or widths can be supported by one of the ledges.

Preferably, according to another embodiment, the medicament container can be a cartridge or a syringe.

Preferably, according to another embodiment, the medicament delivery device can be an injector, an inhaler, or a medical sprayer.

Preferably, according to another embodiment, the injector is an autoinjector, an insulin pen, or a safety syringe.

Preferably, according to another embodiment, the needle guard of the injector can be a user-detachable needle guard or a non-detachable needle guard for an end-user.

Another aspect of the invention provides a method of assembling a medicament delivery device medicament container assembly, the method comprising the following sequence of steps in the following order: providing a tubular body of a medicament container carrier, wherein the tubular body extends along a longitudinal axis between a proximal end and a distal end; providing a medicament container;

providing a proximal cap of the medicament container carrier; inserting the medicament container from the proximal end of the tubular body into the tubular body; and moving the proximal cap of the medicament container carrier relative to the tubular body in a direction transverse to the longitudinal axis to attach the proximal cap to the proximal end of the tubular body.

Preferably, according to another embodiment, the step of moving the proximal cap of the medicament container carrier in the direction transverse to the longitudinal axis comprises the step of linearly moving the proximal cap of the medicament container carrier in the direction transverse to the longitudinal axis.

Preferably, according to another embodiment, the step of moving the proximal cap of the medicament container carrier relative to the tubular body in the direction transverse to the longitudinal axis to attach the proximal cap to the proximal end of the tubular body comprises the step of snap-fitting the proximal cap onto the tubular body.

Preferably, according to another embodiment, the step of moving the proximal cap of the medicament container carrier relative to the tubular body in the direction transverse to the longitudinal axis to attach the proximal cap to the proximal end of the tubular body comprises the step of arranging a ledge extending from an inner surface of the proximal cap so that the ledge contacts a shoulder of the medicament container.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a medicament container carrier.

FIG. 2A schematically shows a perspective view of a tubular body of the medicament container carrier of FIG. 1.

FIG. 2B schematically shows a cross-section view of the tubular body of FIG. 2A.

FIG. 3 schematically shows a perspective view of a proximal cap of the medicament container of FIG. 1.

FIGS. 4A-D schematically show an assembling sequence for assembling a medicament container into the medicament container carrier of FIG. 1.

### DETAILED DESCRIPTION

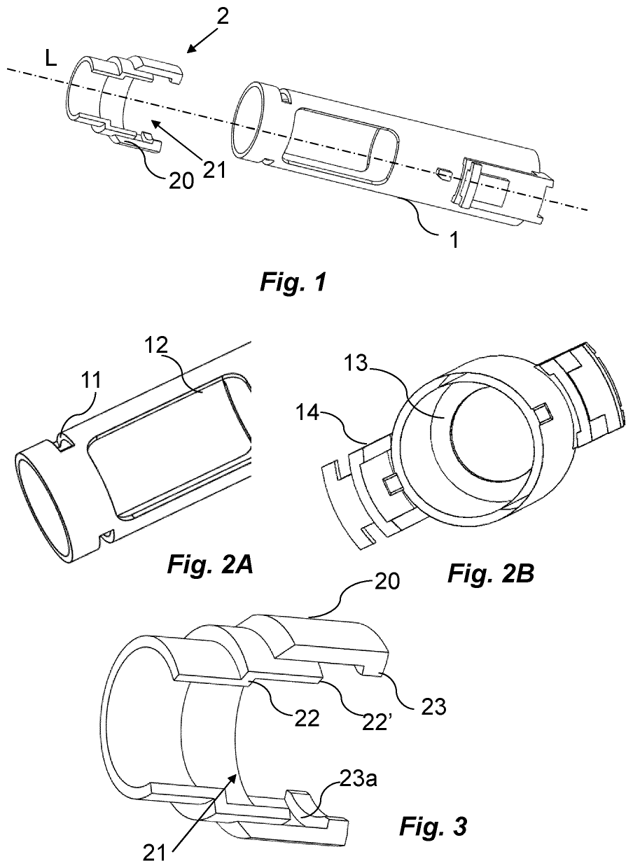

FIGS. 1-4D illustrates a medicament container carrier of a medicament delivery device. The medicament container carrier comprises a tubular body 1 for receiving a medicament container 3 and a proximal cap 2.

The tubular body 1 extends along a longitudinal axis L between a proximal open end and a distal open end. As shown in FIG. 2A, the tubular body 1 comprises a fastener 11, which is preferably arranged closer to the proximal open end than the distal open end. The tubular body 1 optionally comprises a window 12 (this window can be an aperture on a wall of the tubular body or a transparent wall of the tubular body), so that a user of the medicament delivery device can observe the contained medicament within the medicament container 3 that is arranged within the tubular body 1 of the medicament container. Alternatively, a major portion of the tubular body is transparent. In this example, the tubular body doesn't need the window 12. As shown in FIG. 2B, the tubular body 1 optionally comprises a proximally directed surface 13, which is preferably arranged closer to the distal open end than the proximal open end. The proximally directed surface can be defined by a distal ledge extending from an inner surface of the tubular body 1. In one example, the distal ledge is an annular ledge. The proximally directed surface 13 is configured to prevent the medicament container from moving in the distal direction relative to the tubular body 1 when the medicament container is arranged within the tubular body 1. This arrangement of the proximally directed surface 13 is suitable for when the medicament container is configured to be inserted into the medicament container carrier from the proximal open end of the tubular body 1. For example, this arrangement is suitable for when the medicament container is a cartridge. Usually a syringe comprises a flange at a distal end of the syringe, so that if the syringe is configured to be inserted into the tubular body from the proximal open end of the tubular body, the diameter of the tubular body has to be slightly greater than the width of the syringe flange, which thus requires increased size of both of the medicament container carrier and the medicament delivery device. On the other hand, cartridges don't normally have a flange; therefore, the cartridge will be equally suitable for being inserted from the proximal open end of the tubular body 1. The tubular body 1 optionally comprises a flange 14 configured to engage with a part of the medicament delivery device housing, so that the tubular body 1 can be prevented from moving, or can only move in a predetermined space relative to the medicament delivery device housing. Alternatively, the flange 14 can be replaced by a ridge or slot arranged on the outer surface of the tubular body 1.

As shown in FIG. 3, the proximal cap 2 comprises a tubular cap body 20. The tubular cap body 20 extends along the longitudinal axis L between a proximal end and a distal end. A slot 21 extends on the tubular cap body 20 from the proximal end to the distal end. The tubular cap body 20 comprises a ledge 22, 22′ extending from an inner surface of the tubular cap body 20. The ledge 22, 22′ is radial relative to the longitudinal axis L. The ledge 22, 22′ is configured to support a shoulder part 31 of the medicament container 3 of the medicament delivery device. The proximal cap 2 comprises a counter fastener 23 offset relative to the ledge 22, 22′ in the direction of the longitudinal axis L. The proximal cap 2 is configured to be attached to the proximal end of the tubular body 1 through the engagement between the fastener 11 and the counter fastener 23. Furthermore, in one example, the tubular cap body 20 comprises multiple ledges 22, 22′ extending from the inner surface of the tubular cap body 20. If there are multiple ledges 22, 22′, the ledges are offset relative to one another in the direction of the longitudinal axis L, so that the medicament container carrier can be used for medicament containers with different length. For a tubular cap body 20 with multiple ledges 22, 22′; only one such ledge will engage with the shoulder of the medicament container, depending on the length different container. In a preferred example, the tubular cap body has varied inner diameters, so that so that the medicament container carrier can be used for medicament containers with different widths. The shoulders of medicament containers of the medicament delivery device with different length and/or width can be supported by one of the ledges.

In another example, the fastener 11 of the tubular body comprises either a cut-out/recess or a hook; and the counter fastener 23 of the proximal cap 2 is a cut-out (if the fastener of the tubular body is a hook) or a hook (if the fastener of the tubular body is a cut-out). In a preferred example, the fastener 11 is the cut-out/recess, as shown in FIG. 2A. The cut-out/recess is arranged in the wall of the tubular body 1. The cut-out/recess is open in a direction transverse to the longitudinal axis L. In this example, the counter fastener 23 is the hook extending from the tubular cap body 20 in the distal direction relative to the tubular cap body 20. The proximal cap 2 is configured to be attached to the proximal end of the tubular body through a fastener between the hook and the cut-out/recess.

In one example, the cut-out/recess extends in a circumferential direction relative to the longitudinal axis L between a first end and a second end. In a preferred example, a ramp surface is arranged at the first end. The ramp surface inclines from an inside portion of the cut-out/recess to an outer edge of the cut-out/recess. Therefore, the hook can easily enter into the cut-out/recess when the hook is moved linearly relative to the cut-out/recess in a direction transverse to the longitudinal axis L.

In another example, the hook 23 comprises a hook arm and a hook tip 23a. The hook arm extends in the direction of the longitudinal axis L. The hook tip 23a extends from an end of the hook arm in the direction transverse to the longitudinal axis L, as shown in FIG. 3. The hook tip 23a is configured to be arranged within the cut-out/recess. In another example, the hook tip 23a extends in the circumferential direction between a first end and a second end. The hook tip 23 comprises a ramp surface angled relative to the hook arm and arranged at the first end. Thus, the hook can easily enter into the cut-out/recess when the hook is moved linearly relative to the cut-out/recess in a direction transverse to the longitudinal axis L.

In a preferred example, the engagement between the fastener 11 and the counter fastener 23 is a snap-fit engagement. Alternatively, the engagement between the fastener and the counter fastener is a thread screw engagement or a bayonet engagement. In these examples, the fastener and the counter fastener are thread screw or bayonet connections.

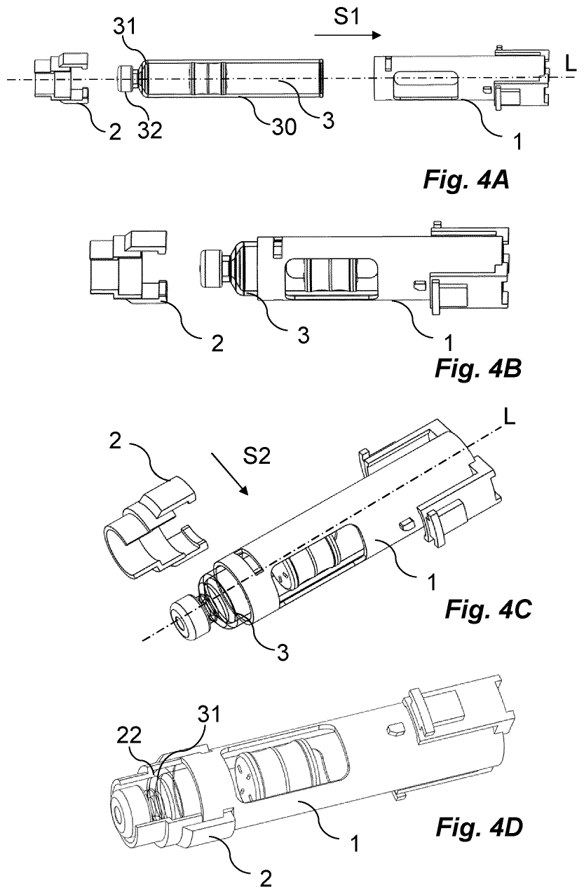

An assembling method for assembling a medicament delivery device medicament container assembly, as shown in FIGS. 4A-4D, will now be described. The method comprises the following steps in the following order:
providing the tubular body 1 of a medicament container carrier,
providing the proximal cap 2 of the medicament container carrier,
inserting the medicament container 3 from the proximal end of the tubular body 1 into the tubular body 1, as shown by the arrow S1 in FIG. 4, and
moving the proximal cap 2 of the medicament container carrier relative to the tubular body 1 in the direction transverse to the longitudinal axis L, as shown by the arrow S2 in FIG. 4C to attach the proximal cap 2 to the proximal end of the tubular body 1.

In this example, a part of a body portion 30 of the medicament container 3, the shoulder 31 of the medicament container 3 and a head 32 of the medicament container 3 are moved into the proximal cap 2 through the slot 21 of the proximal cap 2.

In one example, the step of moving the proximal cap 2 of the medicament container carrier in the direction transverse to the longitudinal axis comprises the step of linearly moving (as shown by arrow S2) the proximal cap 2 of the medicament container carrier in the direction transverse to the longitudinal axis L. In another example, the step of moving the proximal cap 2 of the medicament container carrier 3 relative to the tubular body 1 in the direction transverse to the longitudinal axis L to attach the proximal cap 2 to the proximal end of the tubular body 1 comprises the step of snap-fitting the proximal cap 2 onto the tubular body 1. Alternatively, the step of moving the proximal cap of the medicament container carrier relative to the tubular body in the direction transverse to the longitudinal axis L to attach the proximal cap to the proximal end of the tubular body comprises the step of rotating the proximal cap relative to the tubular body so that the proximal cap is attached to the tubular body by a bayonet or screw thread engagement after the movement of the proximal cap in the direction transverse to the longitudinal axis L.

In another example, the step of moving the proximal cap 2 of the medicament container carrier relative to the tubular body 1 in the direction transverse to the longitudinal axis to attach the proximal cap to the proximal end of the tubular body comprises the step of aligning a ledge extending from the inner surface of the proximal cap 2 to contact the shoulder 31 of the medicament container 3.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A medicament container carrier of a medicament delivery device, the medicament container carrier comprising:
a tubular body for receiving a medicament container, the tubular body extending along a longitudinal axis between a proximal open end and a distal open end, wherein the tubular body comprises a fastener; and
a proximal cap comprising a tubular cap body,
wherein the tubular cap body extends along the longitudinal axis between a proximal end and a distal end, wherein a slot extends on the tubular cap body from the proximal end to the distal end, wherein the tubular cap body comprises a ledge extending from an inner surface of the tubular cap body, wherein the ledge is radial relative to the longitudinal axis, wherein the ledge is configured to support a shoulder part of the medicament container of the medicament delivery device, wherein the proximal cap comprises a counter fastener offset relative to the ledge in the direction of the longitudinal axis, and wherein the proximal cap is attached to the proximal end of the tubular body through an engagement between the fastener and the counter fastener.

17. The medicament container carrier according to claim 16, wherein the fastener is either a cut-out/recess or a hook, and wherein the counter fastener is a hook if the fastener is a cut-out/recess, or the counter fastener is a cut-out/recess if the fastener is a hook.

18. The medicament container carrier according to claim 17, wherein the fastener is the cut-out/recess, wherein the cut-out/recess is arranged in a wall of the tubular body, wherein the cut-out/recess is open in a direction transverse to the longitudinal axis, wherein the counter fastener is the hook extending from the tubular cap body in the distal direction relative to the tubular cap body, and wherein the proximal cap is attached to the proximal end of the tubular body through a fastener between the hook and the cut-out/recess.

19. The medicament container carrier according to claim 17, wherein the cut-out/recess extends in a circumferential direction relative to the longitudinal axis between a first end and a second end, wherein a ramp surface arranged at the first end, and wherein the ramp surface inclines from an inside portion of the cut-out/recess to an outer edge of the cut-out/recess.

20. The medicament container carrier according to claim 17, wherein the hook comprises a hook arm and a hook tip, wherein the hook arm extends the direction of the longitudinal axis, wherein the hook tip extends from an end of the hook arm in the direction transverse to the longitudinal axis, and wherein the hook tip is arranged within the cut-out/recess.

21. The medicament container carrier according to claim 20, wherein the hook tip extends in a circumferential direction between a first end and a second end, and wherein the hook tip comprises a ramp surface angled to the hook arm and arranged at the first end.

22. The medicament container carrier according to claim 16, wherein the engagement between the fastener and the counter fastener is a snap-fit engagement.

23. The medicament container carrier according to claim 16, wherein the tubular body comprises a distal ledge extending from an inner surface of the tubular body.

24. The medicament container carrier according to claim 23, wherein the distal ledge is an annular ledge.

25. The medicament container carrier according to claim 16, wherein the tubular cap body comprises multiple ledges extending from the inner surface of the tubular cap body, and wherein the multiple ledges are offset relative to one another in the direction of the longitudinal axis, so that the shoulders of medicament containers of the medicament delivery device with different lengths can be supported by one of the ledges.

26. The medicament container carrier according to claim 25, the tubular cap body has varied inner diameters, so that the shoulders of medicament containers of the medicament delivery device with different lengths and/or widths can be supported by one of the ledges.

27. A medicament delivery device comprises the medicament container carrier according to claim 16.

28. A method of assembling a medicament delivery device medicament container assembly, the method comprising the following sequence of steps in the following order:
providing a tubular body of a medicament container carrier, wherein the tubular body extends along a longitudinal axis between a proximal end and a distal end;
providing a medicament container;
providing a proximal cap of the medicament container carrier;
inserting the medicament container from the proximal end of the tubular body into the tubular body; and
moving the proximal cap of the medicament container carrier relative to the tubular body in a direction transverse to the longitudinal axis to attach the proximal cap to the proximal end of the tubular body.

29. The method according to claim 28, wherein the step of moving the proximal cap of the medicament container carrier in the direction transverse to the longitudinal axis comprises the step of linearly moving the proximal cap of the medicament container carrier in the direction transverse to the longitudinal axis.

30. The method according to claim 28, wherein the step of moving the proximal cap of the medicament container carrier relative to the tubular body in the direction transverse to the longitudinal axis to attach the proximal cap to the proximal end of the tubular body comprises the step of snap-fitting the proximal cap onto the tubular body.
